(19) United States
(12) Patent Application Publication
(10) Pub. No.: US 2026/0144945 A1
(43) Pub. Date: May 28, 2026

(54) MEDICAL DEVICE WITH ALARM ORGANIZATION

(71) Applicant: Drägerwerk AG & Co. KGaA, Lübeck (DE)

(72) Inventor: Birger LANDWEHR, Lübeck (DE)

(21) Appl. No.: 19/398,221

(22) Filed: Nov. 24, 2025

(30) Foreign Application Priority Data
Nov. 25, 2024 (DE) 10 2024 134 627.2

(51) Int. Cl. A61M 16/00

(52) U.S. Cl. CPC A61M 16/0051; A61M 16/026; A61M 2016/0039; A61M 2205/18

(57) ABSTRACT

A medical device has features for an alarm organization with regard to the time course of a minute volume. The alarm organization features are configured for undershoots (117, 217) and exceedances (118, 218) of a comparison minute volume (208) over a period of time (1000). The features make it possible to avoid overreacting to non-critical situations.

### CROSS REFERENCE TO RELATED APPLICATIONS

This application claims the benefit of priority under 35 U.S.C. § 119 of German Application 110 2024 134 627.21, filed Nov. 25, 2024, the entire contents of which are incorporated herein by reference.

### TECHNICAL FIELD

The present invention relates to a medical device with alarm organization, in particular a medical device suitable for ventilating a living being or a patient, such as an anesthesia machine, an intensive care ventilator, a home ventilator, a mobile emergency ventilator, or ventilators for ventilating premature and/or newborn infants, infants, and children.

### BACKGROUND

During ventilation, certain ventilation parameters, such as ventilation pressures and ventilation frequency, are set and monitored by sensors. If, in the course of such monitoring, events or situations arise that deviate from specified lower and/or upper reference or threshold values, an alarm management system of the medical device typically intervenes to notify the user of the events or situations.

One key parameter that is of great interest to monitor during ventilation is the minute volume (MV). The minute volume is the amount of breathing gas mixture that is individually adjusted for different patients or living beings in order to supply the lungs of these living beings with a sufficient amount of breathing gas mixture. The minute volume is the volume of gas exchange that is exchanged on average during breathing and/or ventilation in a time unit of 1 minute by means of inspiration and expiration. High-quality ventilation therapy for the patient or living being is ensured both by the adjustment of the minute volume and the alarm limits for the minute volume by the medical staff on the medical device and by the sensory monitoring of the alarm limits of the minute volume by the medical device.

A lower alarm threshold value is usually a value of approximately 80% to 90% of the target minute volume MV_Set and can be adjusted by the user to the respective ventilation situation.

Filtering is often used in signal processing to determine the minute volume in order to provide the user with stable and interference-free values as information for the minute volume MV. As suggested by the term “minute volume”, the filters typically have time constants of less than one minute to one minute and take into account several breathing cycles of the living being.

If higher-order filters suitable for filtering are used, e.g., with designs such as Bessel or Butterworth filters, situations may arise in which the event triggering the alarm in the form of a minute volume falling below a lower threshold value becomes effective as an alarm for the user, i.e., audible and/or visible, with a certain delay due to the signal propagation time of the filtering.

From the state of the art there are known possibilities for triggering alarms to a user depending on criteria or depending on certain situations.

For example, U.S. Pat. Nos. 9,962,509 and 9,615,743 show some possibilities for reducing ventilation alarms.

So-called soft threshold methods for alarm reduction with alarm thresholds adjusted during operation are also known, as described, for example, in U.S. Pat. No. 6,754,516, EP2302606 B1, EP2245607 B1, or U.S. Pat. No. 8,515,513.

### SUMMARY

It is an object of the invention to improve alarm organization on a medical device.

This object is attained by features according to this disclosure.

This object is attained by a medical device for ventilating a living being with features according to the invention.

Advantageous embodiments of the invention are derived from the description, claims and drawings and are explained in more detail in the following description with partial reference to the figures.

According to the invention, a medical device is configured to implement and configure an alarm organization. Such a medical device according to the invention can be configured as a ventilator, as an anesthesia machine for ventilating a living being, or as an electroimpedance tomography device (EIT device). According to another aspect of the invention a medical system is provided comprising the 1ventilator or anesthesia machine and a data module configured to implement and configure an alarm organization.

The basic idea of the invention is to adjust an alarm in relation to falling below the minute volume MV on the basis of a predetermined signal time course, which is based on a time course of a comparison minute volume MV_comp. The alarm organization makes it possible to avoid overestimations of non-critical situations.

However, the alarm organization keeps the alarm sufficiently sensitive with regard to critical situations.

To implement the basic idea described above for alarm organization during the ventilation of a living being using a medical device configured for this purpose as a ventilator or an anesthesia machine configured for this purpose, a control unit is suitably configured and provided. According to the invention, during the course of ventilation, a special situation of the minute volume MV is determined in relation to a time course of a comparison minute volume MV_comp.

The determined relationship can then be taken into account for the organization and/or adjustment of alarms to the user, whereby, for example, a control unit is configured to carry out the organization and/or adjustment of alarms to the user.

A medical device configured to implement the idea according to the invention—such as a ventilator—has a control unit, a tube system (line system/hose system), a sensor system, and a breathing gas supply system. The breathing gas supply system comprises a gas conveying unit, an inspiratory dosing unit, and an expiratory valve as a means for performing controlled ventilation of a living being.

The tube system is configured to supply breathing gas quantities to the living being and to remove breathing gas quantities from the living being.

The sensor system may comprise at least one flow rate sensor (volume rate of flow sensor), which is arranged on the breathing gas supply system or on the tube system in such a way as to continuously measure at least one measured value of a flow rate and to provide the control unit with the measured values.

The control unit is configured to coordinate and control the controlled supply and dosing of inspiratory breathing gas quantities and the control of expiratory breathing gas quantities for the ventilation of the living being using the sensor system, the breathing gas supply system, and the tube system. Such a control unit is thus configured to control mechanical ventilation, i.e., for example, to control pressure-controlled ventilation or volume-controlled ventilation.

The control unit can also be configured to control and/or coordinate supportive forms of ventilation based on measured values of ventilation pressures, inspiratory and/or expiratory flow rates or volumes.

These include, for example and in particular, threshold value monitoring of inspiratory and/or expiratory ventilation pressures, threshold value monitoring of carbon dioxide concentrations, threshold value monitoring of oxygen concentrations, threshold value monitoring of inspiratory and/or expiratory flow rates, as well as inspiratory and/or expiratory quantites. In principle, the control unit of a medical device can be configured to control and/or manage the alarm functions of the medical device, ventilator, anesthesia machine, or even a medical imaging device (MRI, CT, radar, ultrasound, EIT, sEMG), for example, an electroimpedance tomography device (EIT).

The control unit is configured to continuously determine a minute volume MV based on the measured values of the flow rate sensor system during ventilation operation. For this purpose, the quantities of inhaled gases and quantities of exhaled gases can be formed from the flow rate measurements provided by the flow rate sensor, which then serve as the basis for the control unit as a basis for determining minute volumes MV. The control unit is further configured to organize alarms based on the minute volume MV during a course of ventilation over a plurality of inspiratory durations.

The control unit is further configured to determine a special situation of the minute volume MV in relation to a time course of a comparison minute volume MV_comp. during the course of ventilation, to take this into account for the organization of alarms, and to carry out an adjustment of an alarm depending on the special situation of the minute volume MV.

If the special situation of the minute volume MV arises in such a way that the minute volume inhaled/exhaled by the living being is lower than the time course of the reference minute volume MV_comp. set, as the threshold, the control unit triggers an alarm with regard to an insufficient minute volume MV for the duration of the special situation.

Such an alarm can be triggered, for example, by providing a control signal to an alarm unit, which is configured to issue a visual and/or acoustic alarm to the user.

In a particular embodiment, the adjustment of the alarm can be organized in such that the control unit is configured to trigger the alarm in such a way that a delay time T_delay is provided until the alarm is actually activated for the user.

In a further preferred embodiment, the control unit can derive the duration of the delay time T_delay from the specified (predefined) signal time course of the comparison minute volume MV_comp. Furthermore, and particularly preferably, the specified signal time course can be configured as a time-filtered signal time course. The specified signal time course can be stored in the control unit itself in a data module (of a data storage/memory) to which the control unit has at least read access.

The control unit may be configured to implement this delay by the delay time T_delay as an adjustment of the alarm depending on the special situation of the minute volume MV. The adjustment can be configured by the control unit in such a way that the expiration of the delay time T_delay is waited for until the actual visual or acoustic alarm is triggered for the user. Taking into account the special situation determined with regard to the minute volume MV, the coordination of the implementation and configuration of the alarm organization according to the invention, with a focus on deviations of the current minute volume MV from the comparison time course of a minute volume MV, can have the advantage that the number of alarms with regard to an insufficient minute volume is limited to those situations of ventilation in which, during the analysis of the measured values of the current minute volume of the living being for a specific time interval after a falling below a threshold value of a minute volume that is too low, no changes in the situation for the living being are to be expected or predicted during the next breaths.

In a preferred embodiment, the control unit may be configured to derive the delay time T_delay as a function of a distance, deviation, relation, or difference between the patient current minute volume MV_pat. and a comparison minute volume MV_comp. and to use this for organizing and/or adjusting an alarm.

In a preferred embodiment, the control unit may be configured to derive the delay time T_delay as a function of a distance between the current minute volume MV_pat. and a comparison minute volume MV_comp, a deviation between the current minute volume MV_pat. and a comparison minute volume MV_comp, a relation between the current minute volume MV_pat. and a comparison minute volume MV_comp, or a difference between the current minute volume MV_pat. and a comparison minute volume MV_comp. and to use this for organizing and/or adjusting an alarm. Such a comparison minute volume MV_comp. may, for example, be stored as a data record in a data storage, for example in the form of a signal-time course or as a table.

In a preferred embodiment, the control unit can be configured to derive the duration of the delay time T_delay from a reference value and to use the delay time T_delay for the organization and/or adjustment of an alarm. The reference value can preferably be based directly or indirectly on the comparison minute volume MV_comp. or index the comparison minute volume MV_comp. Such a reference value may, for example, be stored as a data value in a data record in the data moule of the data storage.

In a preferred embodiment, the control unit may be configured to derive the duration of such a delay time T_delay from a distance, deviation, relation, or difference between the current minute volume MV_pat. and a lower threshold value MV_thres. of the comparison minute volume MV_comp. and to use it for organizing and/or adjusting an alarm. Such a threshold value MV_thres. may, for example, be stored as a data value in a data record in a data storage device.

In a preferred embodiment, the control unit can be configured to determine the relation, the reference value, the lower threshold value MV_thres. or the time course of the comparison minute volume MV_comp. based on a physiological comparison situation of the living being, in particular a typical breathing situation or ventilation situation of the living being and to use it for organizing and/or adjusting an alarm.

Typical ventilation situations can result from the living being's own breathing activity with inhalation and exhalation, as well as from gas flows caused by a ventilator or anesthesia machine from device-controlled inspiration phases and expiration phases.

In a preferred embodiment, the control unit may be configured to apply a state as a physiological comparison (reference) situation which is characterized by at least temporary respiratory arrest (cessation of breathing) of the living being or an apnea situation. A physiological comparison situation of the living being can, for example, be derived from a time course of the minute volume during which the living being breathes only very shallowly, i.e., with a shallow breathing depth or very little breathing. An apnea situation represents a borderline case of very, very shallow breathing by the living being with a transition to the possibility of temporary or prolonged respiratory arrest. An apnea situation therefore indicates that the breathing of the living being is so shallow or low that neither sensor technology using flow measurement or sensor signals from electromyography (sEMG), nor imaging diagnostics of the lungs (electroimpedance tomography, computer tomography, magnetic resonance tomography, sonography) can detect any significant breathing activity in the living being that could cause gas exchange in the living being's lungs. For this purpose, a predetermined time period T_apnea can be described as a maximum time period that elapses as soon as, during continuous recording over time of the breathing and/or ventilation of the living being of a minute volume MV_pat, for example based on volumes determined by integrating measured values of flow rates of inhalation and/or exhalation of the living being, an event of respiratory arrest can be identified as an apnea situation at the end of a signal processing chain with signal amplification and signal filtering during the processing of the measured values of the flow rates. The identification can take place, for example, as soon as the minute volume MV_pat. determined by means of signal processing and signal filtering falls below a predetermined threshold value. A time course of an apnea situation can preferably and, for example, be stored as a data record in the data module of the data storage, for example in the form of a signal-time course or as a table. Such a time course of an apnea situation can serve as the basis for a time course of a comparison minute volume MV_comp.

A determination of the time course of the comparison minute volume MV_comp. based on a physiological comparison situation can, for example, be configured in such a way that a signal course of the minute volume, as it results from a physiological event—such as a typical apnea situation—at the end of the signal processing and signal filtering chain, is used directly as the basis for the comparison minute volume MV_comp. and stored as such a time course in the data storage.

However, it is also possible to determine the comparison minute volume MV_comp. by means of tolerance bands applied to the signal course of the minute volume. Configurations with permissible signal deviations from the signal course of the typical apnea situation can also be stored in tables or as function curves in the in the data module of the data storage.

Different maximum permissible durations can also be incorporated into the signal course of the typical apnea situation itself as variables of the configuration of the predetermined duration T_apnea.

According to a particularly preferred embodiment, the predetermined duration T_apnea can be selected depending on patient demographic criteria, for example in the form of patient categories. Patient categories can be defined, for example, on the basis of a distinction between adult patients (adult) and premature or newborn patients (neonate). The characteristics typical for the respective patient category, such as respiratory rate (RR), minute volume (MV), and tidal volume (VT), can be included in the selection of a predetermined duration T_apnea that is suitable and adapted to the patient category. Patient demographic criteria are, for example, criteria that differentiate between patient types such as premature infants, newborns, infants, toddlers, children, adolescents, and adults based on age. For example, the alarm settings for adults can be configured with greater tolerances than those for infants, since the larger blood volume in the circulatory system of an adult compared to that of an infant means that a possible undersupply of oxygen to areas of the brain occurs much later.

The predetermined duration T_apnea, which is adapted to the patient category, can be used, for example, to coordinate the comparison function MV_comp. to a signal processing configuration which, in terms of the filtering properties (filter type, filter order, cut-off frequencies) for low-pass filtering to determine the time course of the minute volume for adults or newborns, can be configured differently.

In a preferred embodiment, the control unit may be configured to include, in the organization of alarms, the manner in which the fluidic-pneumatic connection of the medical device, in particular a ventilator, is configured via the tube system with the living being, and to include this in the organization and/or adjustment of the alarm.

In normal clinical use, examples of fluidic-pneumatic connections that are common options for connecting to the living being or patient include an endotracheal tube, a nasal mask, or a tracheostoma. Such inclusion can be made in particular with regard to the configuration of the delay time T_delay in order to take into account different orders of magnitude of leakage, such as those that can occur with connections using an endotracheal tube and tracheostoma compared to connections using a nasal mask.

Such inclusion can also be made with regard to the duration of the predetermined time period T_apnea, which is configured as a variable, in order to take into account differences in the signal course of typical apnea situations, also caused by possible leaks, which can occur, for example, when connecting via an endotracheal tube and tracheostoma compared to connecting via a nasal mask.

In a preferred embodiment, the control unit may be configured to include a type of ventilation mode used during ventilation in the organization of alarms. Types of ventilation modes include, in particular, volume-controlled or pressure-controlled ventilation modes as well as ventilation modes that support the spontaneous breathing of the living being (the patient's own breathing).

In the aforementioned embodiments, the data storage device may be assigned to the control unit or configured as an element of the control unit. The data storage device is configured to provide storage of data or information, for example in the form of data records, data sets, or tables. This includes, for example and in particular, information
on threshold values, tolerance bands, durations, waiting times;
on patient categories;
on the fluidic-pneumatic coupling of the living being;
on the activated ventilation mode; and
on comparison values, comparison curves, specifications, specification curves, to be stored and made available to the control unit.

Possible configurations of the data storage device can, for example, be RAM, ROM, EPROM, EEPROM, mobile data carriers (CF or SD card, RFID tag, USB stick).

If, in a particularly preferred variant of the described embodiments, the signal course of the comparison minute volume MV_comp. directly corresponds to a signal course typical of an apnea situation for fluid-pneumatic coupling and for signal processing and signal filtering, such a signal course of the comparison minute volume MV_comp. represents, as it were, a curve of the minute volume which cannot be undershot over the course of time during normal ventilation of a living being, even with very shallow breathing.

Variations in the signal courses of the comparison minute volume MV_comp. can be derived from this direct signal course by means of ratio formation or difference formation. These variations can be advantageous, for example, in order to adjust the time management of alarms with regard to the administration of an insufficient minute volume, for example, an adjustment to the alarm management of the medical device as a whole, in which, in addition to monitoring the minute volume, monitoring of volumes, pressure levels, or flow rates (e.g., VT, P_AW, P_insp, P_exp., PEEP, dV/dt_insp., dV/dt_exp.).

In summary, the present invention advantageously enables the alarms relating to an insufficient minute volume to be adjusted in a suitable and varied manner according to the preferred embodiments, both to the signal processing and signal filtering, to the fluidic-pneumatic coupling, and specifically to patient categories.

In the following, exemplary embodiments of the invention are explained in more detail with reference to the figures, without limiting the generality of the invention. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a structure of a ventilator as the medical device according to the invention;

FIG. 2 is a representation of the minute volume MV (t) over time;

FIG. 3a is a graph of a minute volume time course showing one of two situations; and

FIG. 3b is a graph of a minute volume time course showing another of the two situations.

### DESCRIPTION OF PREFERRED EMBODIMENTS

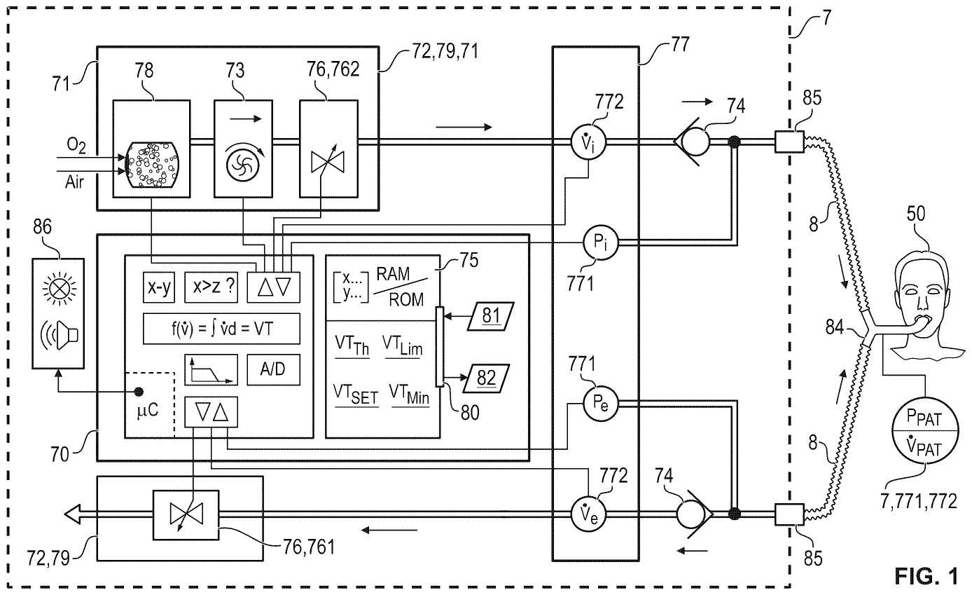

Referring to the drawings, identical elements in FIGS. 1, 2, 3a, and 3b are designated with identical reference numbers in FIGS. 1, 2, 3a, and 3b.

FIG. 1 shows a schematic representation of a medical device configured as a ventilator 7 for ventilating a living being or patient 50. The ventilator 7 and patient 50 are connected via connecting elements 85 to a tube system 8 configured as a breathing tube system with an inspiratory breathing tube for supplying fresh inhalation gases to the patient 50 and an expiratory breathing tube for removing exhaled gases from the patient 50. Ventilator 7 has a sensor system 77 with a pressure sensor system 771 and a flow rate sensor system 772, as well as a dosing unit 71 with a gas mixing unit 78 for providing a breathing gas mixture with defined proportions of air and oxygen, and a gas conveying unit 73.

A control unit 70 with a data module of an associated data storage 75, is provided to coordinate and control the ventilation of the living being 50 and to implement an alarm organization. The control unit 70 is equipped with a computing unit (μC), driver stages, signal processing such as amplifiers (OP amps), filter circuits, A/D converters, and the associated data storage 75, and is configured to control the execution of ventilation by means of the actuators 71, 72, 73, 76, 78, 79, 761, and 762. To perform ventilation and also to trigger alarms, the control unit 70 uses measured values recorded and provided by the sensor system 77, 771, and 772 in combination with criteria such as, in particular, threshold values and default values.

The control unit 70 has the necessary functions and/or program codes in a suitably configured configuration to perform mathematical or logical operations such as difference formation, integral formation, or to perform comparisons with comparison values, threshold values, or tolerance bands in order to be able to organize and/or adjust the alarm as well as the ventilation control.

There is a valve unit 79 controllable by the control unit 70 with actively controllable valves 76, such as in the form of an expiratory valve 761 and an inspiratory dosing valve 762, to effect the dosing of gas quantities to the patient 50 and, in cooperation with passive check valves 74, to determine the direction of the gas flows in the breathing tube system 8. The valve unit 79 and the dosing unit 71 together form a breathing gas supply system 72.

An interface 80 allows data or information 81 to be input to the control unit 70 and/or outputs 82 of data or information from the control unit 70 of data or information can be provided externally from the control unit 70.

The sensor system 77 with pressure sensors 771 and flow rate sensors 772 can be configured for inspiratory, expiratory, and/or patient-related use on a connecting element 84 of the breathing tube system 8, known as the “Y-piece.” The sensor system 77 continuously provides measured values to the control unit 70 for the coordination of ventilation and alarms. From the measured values of the flow sensor 772, the control unit 70 uses temporal integration to determine the volumes supplied to the patient 50 during ventilation and, based on this, a tidal volume VT and, in particular, a minute volume MV and a comparison minute volume MV_comp.

An alarm unit 86 is usually connected to the control unit 70, which is configured to provide information, alarms, and/or signals—in particular with regard to an insufficient minute volume—to a user in an acoustic and/or visual manner. The data storage 75 with the data module is configured to store, hold and provide data or information such as threshold values, comparison values, tolerance bands, default values, signal-time courses, tables, situations, durations, waiting times, preferably specific to different patient categories. The data storage device 75 can be configured to provide a reference value or a lower threshold value MVₜₕᵣₑₛ (MV_thres.) of the minute volume MV. The reference value or the lower threshold value MVₜₕᵣₑₛ (MV_thres.) can preferably be derived from ventilation settings which can be individually adjusted by a user when configuring the ventilation settings for the patient 50 on the ventilator 7 and/or can be preset for patient categories (adults, children, infants, newborns or premature babies) on the ventilator 7. The derivation can be made from set values for tidal volume VT and/or respiratory rate RR. The reference value or the lower threshold value MVₜₕᵣₑₛ (MV_thres.) can also be set directly by the user and/or preset for patient categories (adults, children, infants, newborns or premature babies) on the ventilator 7. In addition to the reference value and/or the lower threshold value MVₜₕᵣₑₛ (MV_thres.) a reference time period can also be stored in the data storage 74, which is indicated as the maximum permissible time period T_apnea_max. and specifies the maximum length of time that respiratory arrest during ventilation of the patient 50 is tolerated by the ventilator 7 and/or the alarm unit 86 before an alarm is triggered for the user.

The time period T_apnea_max. can be set by the user as a time interval and/or preset for patient categories (adults, children, infants, newborns or premature babies) on the ventilator 7.

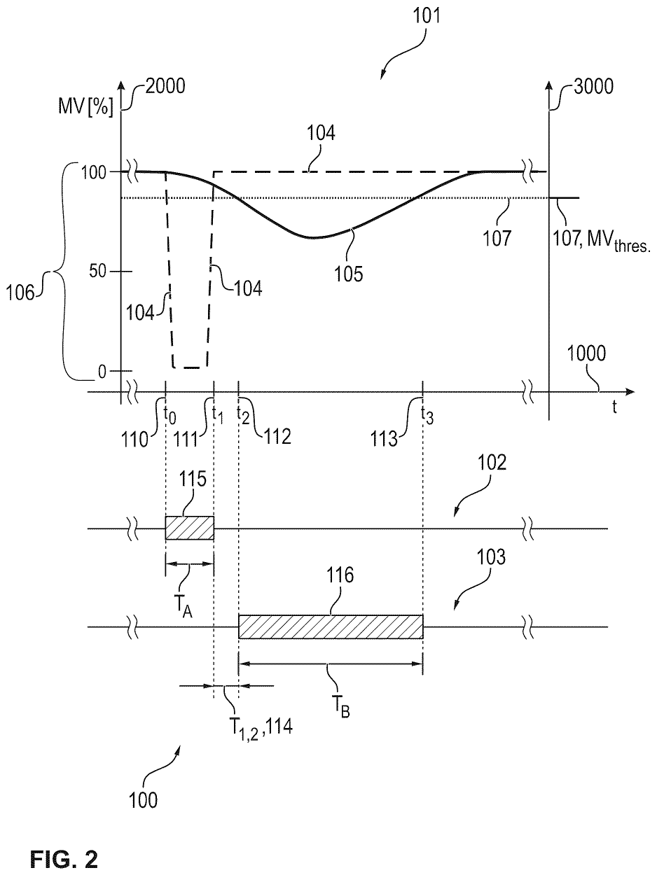

FIG. 2 shows a representation 100 with a time course 101 of the minute volume MV (t) 105 in an exemplary extreme situation of temporary respiratory arrest (apnea) of a patient 50 (FIG. 1) during ventilation by a ventilator 7 according to FIG. 1.

The explanations based on the ventilator 7 (FIG. 1) also cover, in a comparable or identical manner, the technical, physical, or physiological relationships as they are given by performing ventilation of a patient 50 (FIG. 1) as functions of an anesthesia machine. A time axis 1000 is shown, as are Y-axes 2000, 3000. The left Y-axis 2000 of FIG. 100 shows a minute volume MV 101, scaled as a percentage of a target minute volume MV_set (100%).

On the right Y-axis 3000, a scale for a lower threshold value of the minute volume MVₜₕᵣₑₛ (MV_thres._low) 107 is plotted. The lower threshold value of the minute volume MV_thres. _low 107 is shown as a dotted line parallel to the X-axis 1000.

In the time course 101, a signal course MV_data 104—shown as a dashed line—is displayed together with values of a minute volume signal MV_comp. 105—shown as a solid line—which is based on processed values of this signal MV_data 104.

According to this FIG. 2, the minute volume MV_comp. 105 and its time course MV_comp. (t) are obtained by means of data processing and/or signal filtering based on the values of the signal course MV_data 104, for example by the control unit 70 (FIG. 1).

During the course of breathing or ventilation of a patient 50 (FIG. 1), respiratory arrest (apnea) occurs at a point in time t₀ (t_0), which lasts for a period of time until the point in time t₁ (t_1). Over the course of the signal MV_data 104, the respiratory arrest is schematically illustrated as a drop in the signal MV_data 104 from a minute volume level of 100% of a target minute volume MV_set to a level of nearly 0%.

The time difference between the points in time t₀ (t_0) 110 and t₁ (t_1) 111 results in a time duration T_{A} (T_A) 115, as shown schematically in an event history 102.

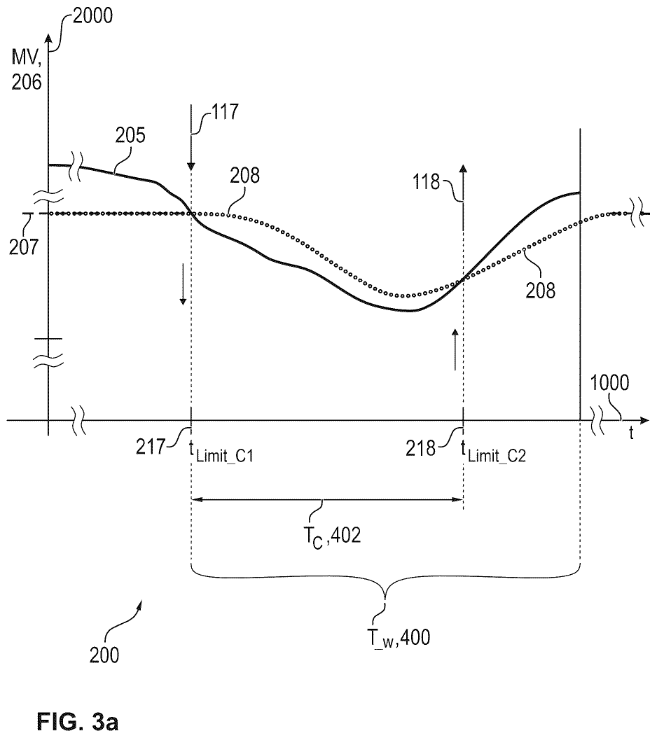

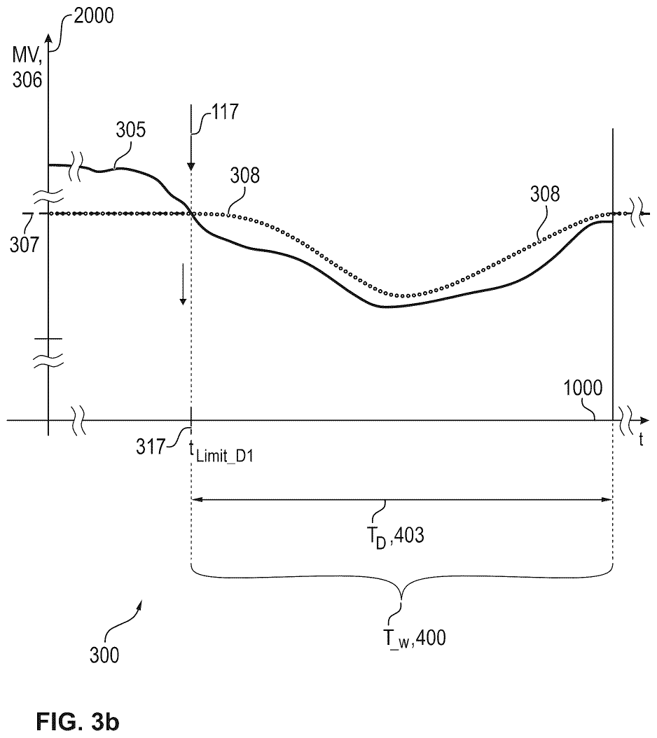

The time course 100 of a minute volume shows how respiratory arrest manifests itself as a significantly delayed drop in the signal MV_comp. 105, 205 (FIGS. 3a, 3b) from a minute volume level of 100% of a target minute volume MV_set for a time duration T_{B} (T_B) 116 shown schematically in an event history 103 to a level of approximately 50%.

In this example illustration 100, there is no drop below 50% because the time interval T_{A} (T_A) 115 is not long enough; in other words, the respiratory arrest had already come to an end before the minute volume signal MV_comp. 105 could have dropped further towards 0%.

The minute volume signal MV_comp. 105 serves as a comparison curve or comparison signal course 208 (FIG. 3a, FIG. 3b) for further explanations of FIGS. 3a and 3b with regard to the alarm organization during monitoring of the patient minute volume MV in the course of ventilation of a living being 50 (FIG. 1).

In FIG. 2, there is filtering of the signal MV_data 104 with a higher-order low-pass filter. This may be, for example, a Bessel filter of order 4, which produces a signal course comparable to that shown in FIG. 2 by signal MV_comp. 105.

If the minute volume MV_comp. 105 is used to determine whether the patient 50 (FIG. 1) is being supplied with an insufficient minute volume MV, the lower threshold value of the minute volume MV_thres._low 107 is used, it follows that the minute volume MV_comp. 105 falls below the lower threshold value MV_thres._low 107 at time t₂ (t_2) 112 and exceeds the lower threshold value MV_thres._low 107 again at time t₃ (t_3) 113. The time difference between times t₂ (t_2) 112 and t₃ (t_3) 113 results in a time duration T_{B} (T_B) 116, as shown schematically in an event sequence 103.

From the two event sequences 102, 103 it becomes clear that the event of respiratory arrest—or situations comparable to respiratory arrest, e.g., situations with shallow breathing—becomes visible much later on the basis of the minute volume signal MV_comp. 105 than would theoretically be possible on the basis of the values of the signal MV_data 104. than would theoretically be possible based on the values of the signal MV_data 104.

From the two event sequences 102, 103 it also becomes clear that the duration of visibility of the event T_{B} (T_B) 116 based on the minute volume MV_comp. 105 lasts more than twice as long as the situation of respiratory arrest that was actually effective in patient 50 (FIG. 1).

The two event sequences 102, 103 also clearly show that the start of event T_{B} (T_B) 116 based on the minute volume MV_comp. 105 begins at time t₂ (t_2) 112, when the situation of respiratory arrest had already actually passed for the patient 50 (FIG. 1).

The start of the event at time t₂ (t_2) 112 occurs a period of time T_{1,2} (T_1,2) 114 after the end of the respiratory arrest situation at time t₁ (t_1) 111.

As shown in this FIG. 2, signal processing and/or signal filtering can result in some effects and situations that can lead to outputs to the user even though an event that may be uncomfortable or dangerous for the patient 50 (FIG. 1) is no longer acute.

These explanations following FIG. 2 illustrate the intention of the present invention, which is to provide a solution for improving the alarm organization on a medical device, in particular the alarm organization on a ventilator or anesthesia machine. With reference to the situation of respiratory arrest (apnea) explained in FIG. 2, FIGS. 3a and 3b show the event sequences 102, 103 and the time sequence 101 of the signal MV_data 104 and the minute volume MV_comp. 105, the configuration of the invention with an improvement in the alarm organization for the minute volume is explained in more detail, and the differences from the current situation according to FIG. 2 and the advantages made possible by the invention are explained in more detail. It should be noted that the situation of respiratory arrest (apnea) during breathing and/or ventilation corresponds to an extreme situation (FIG. 2).

The aim of comfortable, high-quality ventilation for patient 50 (FIG. 1) is to give the patient the opportunity to breathe on their own (spontaneous breathing) during ventilation with supportive forms of ventilation. Therefore, the monitoring of the minute volume MV_comp. 105 as well as possible alarms in the event of an insufficient minute volume MV_comp. 105 in the sense of the present invention are based on a fictitious situation of respiratory arrest (apnea) with regard to the considered properties of signal processing and/or signal filtering.

The extreme situation (FIG. 2) of respiratory arrest (apnea) as actually occurring in patient 50 (FIG. 1) can be avoided by implementing the alarm organization according to the invention in the same way or in an improved way as a theoretically possible implementation of a possible alarm in relation to an insufficient minute volume directly based on values of the signal MV_data 104, with the disadvantages of interference from noise signals and/or noise.

The illustrated extreme situation (FIG. 2) of respiratory arrest (apnea) for a predetermined period of time thus merely represents a kind of comparison function or test function in order to qualify the properties of signal transmission by means of a “step response” for the implemented type and manner of an arrangement and configuration for signal processing and signal filtering in determining a minute volume. The minute volume MV_comp. 105 serves as the basis for a comparison curve or comparison function 208, 308 (FIGS. 3a, 3b) for further explanations of FIGS. 3a and 3b, which can be stored in a data storage 75 (FIG. 1) and can be used by the control unit 70 (FIG. 1) during ventilation to organize alarms relating to a minute volume MV.

FIGS. 3a and 3b show time courses 200, 300 with representations of signal courses 205 (FIG. 3a) and 305 (FIG. 3b) of the minute volume MV_pat. 206 (FIG. 3a) and 306 (FIG. 3b), plotted on the Y-axis 2000 as functions of time over the time axis t 1000. FIGS. 3a and 3b are explained in more detail below in a joint figure description with mutual reference and explanations of the differences between the signal courses 205, 305.

After a current value of the minute volume MV_pat. 205, 305 falls below a lower threshold value MV_thres._low 207 (FIG. 3a) or 307 (FIG. 3b) for the first time during the course of ventilation of a patient 50 (FIG. 1) 205, resulting in an event t_Limit 117, 217, 317 of a threshold value being undershot, a continuous analysis is then carried out over the further course of time for a period of time T_w 400 with the aid of the control unit 70 (FIG. 1) as to the relationship between the subsequent values of the mean minute volume MV_pat. 205, 305 and a comparison function 208, 308.

The comparison function 208, 308 can be selected in such a way that a filtered signal course 105 (FIG. 2) of a situation of respiratory arrest lasting for a predetermined period of time 115 (FIG. 2) serves directly as the comparison function 208, 308, or a function derived from this signal course 105 (FIG. 2) serves as a comparison function 208, 308.

The comparison function 208 is used from a point in time t_{Limit_T1} (t_Limit_C1) 217 for performing the subsequent analyses with current values of the minute volume MV_pat. 205.

The comparison function 308 is used from a point in time t_{Limit_D1} (t_Limit_D1) 317 for performing the subsequent analyses with current values of the minute volume MV_pat. 305.

If, as shown in FIG. 3a, after the initial undershoot of t_Limit_C1 217 of a lower threshold value MV_thres._low 207, the current values of the minute volume MV_pat. 205 temporarily and/or for a predetermined time interval fall below the comparison function 208—for example, until a time t_{Limit_C2} (t_Limit_C2) 218 with threshold value exceedance 118—, the continuous analysis by the control unit 70 (FIG. 1) shows that a situation C exists in which the patient 50 (FIG. 1) is temporarily being administered an insufficient minute volume.

As a result, an alarm is activated for a period of time T_{C} (T_C) 402 for the user with regard to an insufficient minute volume as long as situation C exists.

This situation C indicates that although patient 50 (FIG. 1) was temporarily administered an insufficient minute volume, a stable ventilation situation was achieved again after a certain time, thus enabling the alarm relating to an insufficient minute volume to be deactivated for the user.

If, as shown in FIG. 3b, after the initial undershoot of t_Limit_D1 317 of a lower threshold value MV_thres._low 307, the current values of the mean minute volume MV_pat. 305 permanently fall below the comparison function 308 over time, the continuous analysis by the control unit 70 (FIG. 1) shows that a situation D exists in which the patient 50 (FIG. 1) is being administered an insufficient minute volume.

As a result, an alarm is activated for a period of time T_{D} (T_D) 403 for the user with regard to an insufficient minute volume as long as situation D exists. This situation D indicates that the patient 50 (FIG. 1) is being administered an insufficient minute volume and the alarm therefore cannot be deactivated, at least in the time scale according to this FIG. 3b.

According to an advantageous aspect of the invention, a medical system is provided comprising a ventilator 7, which may be part of an anesthesia device, for ventilating the patient (a living being) 50, in combination with the data module 75. The ventilator 7 comprises the breathing gas supply system 72, which comprises a means for performing controlled ventilation of a patient and includes a gas conveying unit (blower) 73, the inspiratory dosing unit 71 and the expiratory valve 76, 761. The ventilator 7 also includes the tube system 8 to supply breathing gas quantities to the patient 50 and to convey breathing gas quantities away from the patient 50. The sensor system 77, 771, 772, comprising the flow rate sensor system, is arranged operatively connected to the breathing gas supply system 72 and/or the tube system 8 and is configured to continuously detect one or more flow rate measured values and to provide the flow rate measured values to the control unit as measured values. The control unit 70 is configured, in cooperation with the sensor system 77, 771, 772, the breathing gas supply system 72 and the tube system 8, to control a supply and dosage of inspiratory breathing gas quantities and to control expiratory breathing gas quantities for ventilation the patient 50. The control unit 70 is configured to determine a patient current minute volume MV_pat. 205, MV_pat. 305, based on the measured values of the flow rate sensor system 77, 771, 772. The data module 75 is operatively connected to the control unit 70. The data module 75 comprises the apnea special situation comparison data, which comprises a comparison minute volume relation, a comparison minute volume reference value, a comparison minute volume lower threshold value or a comparison minute volume time course 208, 308, based on an apnea comparison situation of the living being for an apnea reference situation duration (period of time) T_w 400. The control unit is configured to determine from the patient current minute volume MV_pat. 205, MV_pat. 305 an apnea situation during a course of ventilation, and to compare the patient current minute volume MV_pat. 205, MV_pat. 305 to the apnea special situation comparison data (comparison minute volume time course 208, 308) and to adjust an alarm organization based on the comparison of the patient current minute volume MV_pat. 205, MV_pat. 305 to the apnea special situation comparison data (comparison minute volume time course 208, 308).

The control unit 70 is configured to adjust the alarm organization by determining a delay time T_delay and to trigger an alarm such that the delay time T_delay elapses before the activating the alarm. The delay time may be determined as a function the patient current minute volume MV_pat. 205, MV_pat. 305 in relation to a lower minute volume threshold value MV_thres. The delay time may be determined as a function of the patient current minute volume MV_pat. 205, MV_pat. 305 in relation to the apnea special situation comparison data comparison minute volume time course 208, 308). The control unit may be configured to determine the delay time based on the apnea reference situation duration T_w 400.

As noted above, the apnea reference situation data may comprise the comparison minute volume time course 208, 308 for an apnea reference situation duration. The control unit may be configured to trigger an alarm based on the patient current minute volume MV_pat. 205, MV_pat. 305 undershooting the comparison minute volume time course 208, 308 for a duration longer that the apnea reference situation duration T_w 400. or may be configured to activate the alarm based on the comparison and after a delay time T_delay such that the delay time T_delay elapses before the activating the alarm. The apnea situation during a course of ventilation may be determined based on the patient current minute volume signal MV_pat. 205, MV_pat. 305, determined by processing and/or signal filtering, falling below a predetermined threshold value MVthres (MV_thres.). While specific embodiments of the invention have been shown and described in detail to illustrate the application of the principles of the invention, it will be understood that the invention may be embodied otherwise without departing from such principles.

### Reference Number List

7 Ventilator, medical device
8 Breathing tube system, line system
50 Patient, living being
70 Control unit, μC
71 Dosing unit
72 Breathing gas supply system
73 Gas conveying unit (blower)
74 Check valves, passive valves
75 Data storage (RAM, ROM)
76, 761, 762 Active valves, expiratory valve, inspiratory dosing valve
77, 771, 772 Sensors, pressure sensors, flow rate sensors
78 Gas mixing unit
79 Valve unit
80 Interface
81 Inputs
82 Output
84 Connecting element (Y-piece)
85 Connecting elements
86 Alarm unit
100, 200, 300 Time courses of minute volumes and events
101, 201, 301 Signal/time diagrams of minute volume MV(t)
102, 203 Time courses of events
104 Signal (MV_data.)
105, 205 Minute volume (MV) signal (MV_comp.)
106 Minute volume, scaled to MV_set in percent [%]
107, 207, 307 Minute volume threshold value, MV_thres._low
110 Time t₀ (t_0), start of respiratory arrest
11 Time t₁ (t_1), end of respiratory arrest
112 Time t₂ (t_2)
113 Time t₃ (t_3)
114 Time delay t_1,2
115, 116 Event durations T_{A} (T_A), T_{B} (T_B)
117 Event with threshold value undershoot
118 Event with threshold value exceeded
205 MV signal (MV_pat.) in the respiratory pattern, situation C
206, 306 Unscaled representations of minute volume MV
208, 308 Comparison function
217 t_Limit_C1; Time of the first threshold violation, situation C
218 t_Limit_C2; Time when the comparison function is exceeded, situation C
305 MV signal (MV_pat.) in the respiratory pattern, situation D
317 t_Limit_D1; Time of first threshold undershoot, situation D
400 Observation period T_w
402 T_{C} (T_C), duration of falling below the comparison function, situation C
403 T_{D} (T_D), duration of falling below the comparison function, situation D
10 Time axis, X-axis, abscissa
2000, 3000 Ordinates, Y-axes

What is claimed is:

1. A medical device with alarm organization, the medical device for ventilating a living being, the medical device comprising:
a control unit;
a breathing gas supply system comprising a means for performing controlled ventilation of a patient, which comprises a gas conveying unit, an inspiratory dosing unit and an expiratory valve;
a tube system configured to supply breathing gas quantities to the living being and to convey breathing gas quantities from the living being; and
a sensor system comprising a flow rate sensor system which is arranged operatively connected to the breathing gas supply system and/or the tube system and is configured to continuously detect one or more flow rate measured values and to provide the flow rate measured values to the control unit as measured values,
wherein the control unit is configured, in cooperation with the sensor system, the breathing gas supply system and the tube system, to control a supply and dosage of inspiratory breathing gas quantities and to control expiratory breathing gas quantities for ventilation of the living being,
wherein the control unit is configured to determine a minute volume based on the measured values of the flow rate sensor system,
wherein the control unit is configured to carry out an organization of alarms during a course of ventilation over a plurality of inspiratory time durations, wherein the alarms relate to the minute volume, and
wherein the control unit is configured to determine, during the course of ventilation, a special situation of the minute volume in relation to a time course of a comparison minute volume and the organization of alarms is based on the special situation such that the control unit is configured to adjust an alarm depending on the special situation of the minute volume.

2. A medical device according to claim 1, wherein the control unit is configured to determine a delay time and to adjust the alarm to delay activating the alarm by the delay time.

3. A medical device according to claim 2, wherein the delay time is determined as a function of a distance, a deviation, a relation, and or a difference of a current minute volume in relation to a lower minute volume threshold value.

4. A medical device according to claim 2, wherein the delay time is determined as a function of a distance, a deviation, a relation, or a difference of a current minute volume in relation to the comparison minute volume.

5. A medical device according to claim 2, wherein the control unit is configured to determine the delay from a reference value, wherein the reference value is based directly or indirectly on the comparison minute volume or indicates the comparison minute volume.

6. A medical device according to claim 2, wherein the control unit is configured to determine the delay time from a distance, a deviation, a relation or a difference between a current minute volume relative to a lower threshold value of the comparison minute volume and the determined the delay time to the organization of alarms and/or to the adjustment of an alarm.

7. A medical device according to claim 1, wherein the control unit is configured to derive a comparison minute volume relation, a comparison minute volume reference value, a comparison minute volume lower threshold value or a comparison minute volume time course based on a physiological comparison situation of the living being, a typical breathing situation of the living being or a ventilation situation of the living being, and to apply the derived comparison minute volume relation, the comparison minute volume reference value, the comparison minute volume lower threshold value or the comparison minute volume time course to the organization of alarms and/or to the adjustment of an alarm.

8. A medical device according to claim 7, wherein the control unit is configured to apply a state, which is characterized by at least temporary respiratory arrest of the living being or an apnea situation, as the physiological comparison situation.

9. A medical device according to claim 1, wherein the control unit is configured to include patient categories, patient demographic criteria, or patient-specific criteria to the organization of alarms and/or to the adjustment of an alarm.

10. A medical device according to claim 1,
wherein the control unit is configured to include the configuration of the fluidic-pneumatic connection of the medical device, which is configured as a ventilator, via the tube system with the living being in the organization of alarms and/or the adjustment of an alarm, and/or
wherein the control unit is configured to include a ventilation mode used during the course of in the organization of alarms and/or the adjustment of an alarm.

11. A medical system comprising:
a ventilator device or anesthesia device for ventilating a living being, the ventilator device or anesthesia device comprising:
a breathing gas supply system comprising a gas conveying unit, an inspiratory dosing unit and an expiratory valve;
a tube system configured to supply breathing gas quantities to the living being and to convey breathing gas quantities from the living being; and
a sensor system comprising a flow rate sensor system which is arranged operatively connected to the breathing gas supply system and/or the tube system and is configured to continuously detect one or more flow rate measured values and to provide the flow rate measured values to the control unit as measured values; and
a control unit, which is configured, in cooperation with the sensor system, the breathing gas supply system and the tube system, to control a supply and dosage of inspiratory breathing gas quantities and to control expiratory breathing gas quantities for ventilation of the living being, wherein the control unit is configured to determine a minute volume based on the measured values of the flow rate sensor system; and
a data module operatively connected to the control unit, the data module comprising apnea special situation comparison data, which comprises a comparison minute volume relation, a comparison minute volume reference value, a comparison minute volume lower threshold value or a comparison minute volume time course based on a living being apnea comparison situation for an apnea reference situation duration,
wherein the control unit is configured to determine from a patient current minute volume an apnea situation during a course of ventilation, and to compare the patient current minute volume to the apnea special situation comparison data and to adjust an alarm organization based on the comparison of the patient current minute volume to the apnea special situation comparison data.

12. A medical system according to claim 11, wherein the control unit is configured to adjust the alarm organization by determining a delay time and to trigger an alarm such that the delay time elapses before the activating the alarm.

13. A medical system according to claim 12, wherein the delay time is determined as a function the patient current minute volume in relation to a lower minute volume threshold value.

14. A medical system according to claim 12, wherein the delay time is determined as a function of the current minute volume in relation to the apnea special situation comparison data.

15. A medical system according to claim 12, wherein the control unit is configured to determine the delay time based on the apnea reference situation duration.

16. A medical system according to claim 11, wherein the apnea reference situation data comprises the comparison minute volume time course for the apnea reference situation duration and wherein the control unit is configured to trigger an alarm based on the patient current minute volume undershooting the comparison minute volume time course for a duration longer that the apnea reference situation duration or to activate the alarm, based on the comparison of the patient current minute volume to the apnea special situation comparison data, after a delay time such that the delay time elapses before the alarm is issued.

17. A medical system according to claim 16, wherein the apnea situation during a course of ventilation is determined based on the patient current minute volume signal, which is processed and/or signal filtered, falling below a predetermined threshold value.

18. A medical system according to claim 17, further comprising an alarm unit wherein the control unit is configured to generate a control signal to the alarm unit, which is configured to issue a visual and/or acoustic alarm.

19. A medical system according to claim 11, wherein the control unit is configured to include patient categories, patient demographic criteria, or patient-specific criteria to the organization of alarms and/or to the adjustment of an alarm.

20. A medical system according to claim 11,
wherein the control unit is configured to include the configuration of the fluidic-pneumatic connection of the medical system, which is configured as a ventilator, via the tube system with the living being in the organization of alarms and/or the adjustment of an alarm, and/or
wherein the control unit is configured to include a ventilation mode used during the course of in the organization of alarms and/or the adjustment of an alarm.
